(19) United States
(12) Patent Application Publication
(10) Pub. No.: US 2005/0008666 A1
(43) Pub. Date: Jan. 13, 2005

(54) Extracts of nonfruiting nonphotosynthetic filamentous bacteria for strengthening keratin materials

(75) Inventors: Christophe Lalaudiere, Tours (FR); Richard Martin, Rochecorbon (FR)

Correspondence Address: BURNS DOANE SWECKER & MATHIS L L P, POST OFFICE BOX 1404, ALEXANDRIA, VA, 22313-1404, US

(73) Assignee: L'OREAL, PARIS (FR)

(21) Appl. No.: 10/848,080

(22) Filed: May 19, 2004

(30) Foreign Application Priority Data
May 19, 2003 (FR) 03/05974

(51) Int. Cl. A61K 45/00; A61K 7/04; C12N 1/20

(52) U.S. Cl. 424/401; 424/93.4; 435/252.3

(57) ABSTRACT

Extracts of nonfruiting (nonfructifying) nonphotosynthetic filamentous bacteria, notably the bacterium Vitreoscilla filiformis, are well suited for improving the structure of keratin materials, especially for (i) hardening the nails, increasing their thickness, stimulating their rate of growth and/or of regrowth, and increasing their resistance to breaking and/or to splitting; or (ii) increasing the elasticity and/or maintaining the shaping of the hair and/or of the eyelashes; or (iii) for shaping and/or styling the hair, or for the care of and/or for making up the eyelashes or the nails.

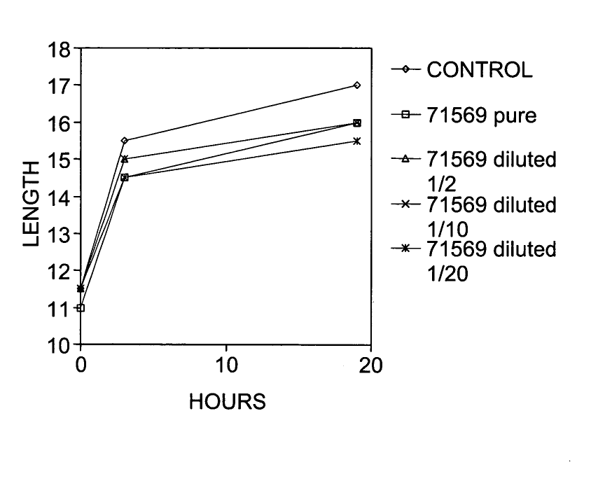

### CROSS-REFERENCE TO PRIORITY APPLICATION

This application claims priority under 35 U.S.C. § 119 of FR 03/05974, filed May 19, 2003, hereby expressly incorporated by reference and assigned to the assignee hereof.

### BACKGROUND OF THE INVENTION

### Technical Field of the Invention

The present invention relates to the formulation, in a cosmetic composition, of an effective amount of at least one extract of a nonfruiting nonphotosynthetic filamentous bacterium, as an agent for improving the structure of keratin materials.

In particular, this invention is directed especially towards (i) hardening the nails, increasing their thickness, stimulating their rate of growth and/or of regrowth, increasing their resistance to breaking and/or to splitting and improving their general condition; or (ii) increasing the elasticity and/or maintaining the shaping of the hair and/or of the eyelashes.

The nonfruiting (nonfructifying) nonphotosynthetic filamentous bacterium is preferably Vitreoscilla filiformis.

The present invention also relates to particular cosmetic compositions suitable, respectively, for shaping and/or styling the hair, or for the care of and/or for making up the eyelashes or the nails, comprising said bacterial extract, and also to a cosmetic process for shaping and/or styling the hair, or for the care of and/or for making up the eyelashes or the nails.

### DESCRIPTION OF BACKGROUND AND/OR RELATED AND/OR PRIOR ART

Keratins are fundamental compounds of the skin, the hair and the nails. These water-insoluble fibrous molecules contribute to the shape of the hair and of the nails and also to their elasticity and to their strength.

It is well known that the nails frequently exhibit structural and consistency defects which may be of various origin, and in particular related to the individual's own metabolism, to his or her living conditions, to his or her dietary habits, to his or her age, and to his or her states of fatigue or of overwork.

These defects may also appear under the effect of actions which erode, for example as a result of prolonged or repeated exposure to detergents, to solvents, to chemical products, in particular those in household use, to moist or dry, hot or cold atmospheres or to exposure to UV radiation.

These defects in structure and consistency have the effect of making the surface of the nails unsightly, and this can be a source of embarrassment and of recurrent inconvenience.

Keratin also plays a role in the elasticity and the shape of the hair fiber. In certain cases, the hair can appear lank and the hairstyle can appear not to hold; these defects in structure may be related, as for the nails, to the individual's own metabolism, to his or her living conditions, to his or her dietary habits, to his or her age, and to his or her states of fatigue or of overwork.

There is therefore a need to find products capable of modifying the physicochemical properties of keratins and thus of influencing the elasticity and/or the strength of keratin materials.

According to this invention, the term “keratin materials” is intended to mean in particular the hair, the eyelashes and the nails.

### SUMMARY OF THE INVENTION

It has now unexpectedly been determined that extracts of nonfruiting nonphotosynthetic filamentous bacteria have an affinity for keratin and are capable, after application to locks of hair or to the nails:
of prolonging the hold or shaping of the hairstyle, by maintaining the curled effect of a lock of hair for a longer period of time and by increasing its elasticity;
of hardening the nails, increasing their resistance to breaking and/or to splitting, increasing the thickness of the nails, stimulating the growth and/or regrowth of the nails, and thus improving their overall appearance and the retention of nail varnishes.

The present inventors have previously shown the immunomodulatory and soothing effect of said bacterial extract and have described cosmetic compositions for eyelashes or hair comprising an extract of a nonfruiting nonphotosynthetic filamentous bacterium combined with an organic UV-screening agent (WO 02/056858), or compositions for the hair comprising said extract combined with a product having an irritant effect (EP-0-761,204). However, no reference of any kind is made to an effect on the structure of keratin materials, nor any description given of the particular compositions for the hair, eyelashes and nails which are subjects of the present invention, implementing this effect. Moreover, the compositions of the prior art preferably use the supernatant from said extract and not the cell extract, as preferred in the context of this invention.

EP-0-876,813 (L'Oréal) concerns a composition comprising an effective amount of a culture medium from at least one nonphotosynthetic filamentous bacterium, which is not the subject of the present invention relating to compositions comprising an extract of the biomass of the bacterium (cell extract or supernatant from said extract).

The present invention therefore features the formulation, in a cosmetic composition, of an effective amount of at least one extract of a nonfruiting nonphotosynthetic filamentous bacterium, as an agent for improving the structure of keratin materials.

The expression “agent for improving the structure” of keratin materials is intended to mean an agent for improving the physical and/or mechanical properties of keratin materials, in particular an agent capable of promoting the growth, and/or the resistance to tension and/or the elasticity of said keratin materials.

According to a first embodiment, when applied to the nails, said bacterial extract is intended to harden the nails and/or to increase their resistance to breaking and/or to splitting.

In particular, the use of this bacterial extract according to the invention makes it possible to reduce the brittleness of weakened nails, and in particular striated, cracked or supple nails and nails having a tendency to split. The nails treated with the bacterial extract according to the invention are therefore harder and stronger and therefore less brittle, and may no longer split and/or no longer crack.

The overall appearance of the nail is thus improved. In particular, the nail treated with a composition containing said bacterial extract is smoother, shinier, less damaged, less split and less brittle.

The invention also features the formulation, in a cosmetic composition, of an effective amount of at least one extract of a nonfruiting nonphotosynthetic filamentous bacterium, said bacterial extract being intended to increase the thickness of the nails.

It also features the formulation, in a cosmetic composition, of an effective amount of at least one extract of a nonfruiting nonphotosynthetic filamentous bacterium, said bacterial extract being intended to stimulate growth and/or regrowth of the nails.

In particular, the compositions for nails based on this extract will be advantageous for promoting regrowth of the nails when said nails have been torn off in an accident or subsequent to a surgical intervention (ingrown nail for example).

The invention also features the formulation, in a cosmetic composition, of an effective amount of at least one extract of a nonfruiting nonphotosynthetic filamentous bacterium, said bacterial extract being intended to prolong the retention of nail varnishes. The varnish is thus less crumbly and more long lasting.

According to another embodiment, when applied to the hair and/or to the eyelashes, said bacterial extract or said composition is intended to increase elasticity and/or to maintain the shaping of the hair and/or of the eyelashes.

This shaping may be due:
for the hair, to a hairstyling or to semi-permanent or permanent reshaping;
for the eyelashes, to curling in the presence of a combination of cosmetic compounds conferring good curling properties on the eyelashes.

### DETAILED DESCRIPTION OF BEST MODE AND SPECIFIC/PREFERRED EMBODIMENTS OF THE INVENTION

The technique for carrying out permanent reshaping of the hair entails, in a first step, in opening the disulfide bonds of the keratin (cystine) using a composition containing a reducing agent (reducing step), and then, after having preferably rinsed the hair, in reconstituting, in a second step, said disulfide bonds by applying an oxidizing composition (oxidizing step, also referred to as fixing step) so as to give the hair the desired shape. This technique makes it possible indifferently to make the hair wavy or to relax it or to remove its curls, it being possible, as appropriate, for the hair to be placed under tension during or outside the reshaping process.

The presence of the bacterial extract according to the invention in hair compositions for permanent reshaping will advantageously make it possible to decrease the concentration of active ingredients intended for the permanent reshaping effect, it being possible for said active ingredients to have harmful effects (decrease in breaking strength, responsible for the coarse appearance of the hair).

Strengthening of the keratin of the hair can also contribute to:
protecting and repairing the hair fiber damaged by UV radiation or chemical treatments (dyeing, perm, etc.) for example;
improving the surface finish of the hair (structure and radiance of the hair);
better adsorption of water and facilitated swelling, giving the hair more volume.

The bacterial extract which can be used in the context of the invention is prepared from nonphotosynthetic filamentous bacteria as defined according to the classification of Bergey's Manual of Systematic Bacteriology (Vol. 3, sections 22 and 23, 9th edition, 1989), among which mention may be made of bacteria belonging to the order Beggiatoales, and more particularly bacteria belonging to the genera Beggiatoa, Vitreoscilla, Flaxithrix or Leucothrix.

Among the bacteria which can be used, mention may be made, for example, of:
Vitreoscilla filiformis (ATCC 15551)
Vitreoscilla beggiatoides (ATCC 43181)
Beggiatoa alba (ATCC 33555)
Flexithrix dorotheae (ATCC 23163)
Leucothrix mucor (ATCC 25107)
Sphaerotilus natans (ATCC 13338)

An extract of Vitreoscilla filiformis (ATCC 15551) will preferably be used.

According to the invention, the term “bacterial extract” is intended to mean an extract of the bacterial biomass or any active fraction of said extract, in particular:

(i) bacterial cells isolated from the culture medium, which have been concentrated, for example by centrifugation (nonstabilized cell extract); or

(ii) bacterial cells which have been concentrated (i), and then subjected to an operation to rupture the bacterial cell envelopes, by any means known to those skilled in the art, such as by the action of ultrasound or, preferably, autoclaving (stabilized cell extract). The term “envelopes” is intended to mean bacterial wall and, optionally, the underlying membranes;

(iii) the supernatant obtained by filtration of the stabilized cell extract (ii), or any active fraction of said extract. The term “active” fraction of said extract is intended to mean any fraction capable of strengthening and/or improving the keratin materials according to the invention.

This active fraction can be obtained by conventional methods of fractionation, such as extraction in the presence of a solvent, selective precipitation or tangential ultrafiltration (TUF), for example.

These extracts or fractions can be conserved, for example, by freezing said extracts or said fractions, and can be used after thawing.

In the remainder of the description, reference will more simply be made to a bacterial “cell extract” ((i) and (ii)) or to a “supernatant” of said extract (iii).

In addition, use will preferably be made, in the context of the invention, of a bacterial “cell extract” (in form (i) or (ii)) or an active fraction of said extract.

To prepare the bacterial extract according to the invention, said bacteria can be cultured according to the methods known to those skilled in the art, or reference may be made in particular to the description in WO-A-93-00741. A cell extract is obtained, from which the supernatant can be separated, for example by filtration and centrifugation. The extract can be used in aqueous form or in lyophilized form. The protocol is described in greater detail in Example 1 hereinafter.

This bacterial extract can be refractionated and used pure or diluted to various concentrations.

According to a preferred embodiment, the bacterial extract or an active fraction of said extract used according to the invention is in an amount of between 0.01% and 100% of the total weight of the composition, preferably between 0.05 and 10% of the total weight of the composition, and even more preferably between 0.1 and 5% of the total weight of the composition.

Of course, those skilled in the art will be able to adjust the concentration of said extract according to the method of application (the topical route) and the use on hair and/or eyelashes or nails, on the basis of the general knowledge in the corresponding cosmetics fields.

The invention also relates to cosmetic compositions of formulations suitable, respectively, for shaping and/or styling and/or maintaining the hair, or for the care of and/or for making up the eyelashes or the nails, containing at least an effective amount of a bacterial extract as defined above, in a cosmetically acceptable medium compatible with keratin materials.

In general, a medium compatible with keratin materials can be anhydrous or aqueous; it can thus comprise an aqueous phase and/or a fatty phase, in particular in the compositions for the eyelashes, comprising at least one fatty substance chosen from oils, waxes, pasty fatty substances, and mixtures thereof.

The term “aqueous phase” is intended to mean water or a mixture of water and of hydrophilic organic solvent(s) such as alcohols, and in particular linear or branched lower monoalcohols containing from 2 to 5 carbon atoms, such as ethanol, isopropanol or n-propanol, and polyols such as glycerol, diglycerol, propylene glycol, sorbitol, pentylene glycol and polyethylene glycols, or else C₂ ethers and C₂-C₄ aldehydes which are hydrophilic.

The water or the mixture of water and of hydrophilic organic solvents can be present in the composition according to the invention in an amount ranging from 0.1% to 99% by weight relative to the total weight of the composition, and preferably from 10% to 80% by weight.

The compositions can also comprise conventional cosmetic additives such as, for example: a film-forming polymer, organic solvents, waxes, oils, fillers, ceramides, plasticizers, surfactants, thickeners, humectants, moisturizers, softeners, coloring materials, pigments, preservatives, fragrances, and mixtures thereof.

It is also possible to use specifically in the hair compositions, reducing agents for permanent reshaping of the hair, or fixing polymers for styling and/or maintaining the hair.

The hair compositions may also comprise surfactants, conditioners or treating agents, ceramides, film-forming polymers, thickeners and a propellant for the packaging of lacquers.

The compositions for coating the eyelashes, which generally comprise waxes, oils, fillers and surfactants, will also comprise according to the invention at least one film-forming polymer capable of conferring good curling properties on the eyelashes.

They may also comprise plasticizers or at least one agent for promoting curling of the eyelashes, such as a dispersion of multiphase particles (containing a soft phase and a rigid phase) as described by the applicant in WO 03/007898. The particles containing rigid and soft phases can be present in the composition in an amount ranging from 0.1% to 70% by weight of particle solids, relative to the total weight of the composition, preferably ranging from 0.5% to 55% by weight, and preferentially ranging from 1% to 40% by weight.

The compositions for the nails according to the invention comprise in particular water or a mixture of water and of hydrophilic organic solvents, and at least one film-forming polymer.

Nacreous pigments will preferably be used in the compositions intended to be applied to the nails.

The term “film-forming polymer” is intended to mean a polymer capable of forming, on its own or in the presence of an auxiliary agent for film formation, a continuous and adhesive film on a support, in particular on the keratin materials. Among the film-forming polymers which can be used in the composition of the invention, mention may be made of synthetic polymers, of the free-radical type or of the polycondensate type, polymers of natural origin, and mixtures thereof. As film-forming polymers, mention may in particular be made of acrylic polymers, polyurethanes, polyesters, polyamides, polyureas, and cellulose-based polymers such as nitrocellulose.

The film-forming polymer may be solubilized or dispersed in the form of particles in the cosmetically acceptable medium of the composition.

When the composition, in particular in the form of a nail varnish, comprises an organic solvent medium, the film-forming polymer can be chosen in particular from alkyd, acrylic and/or vinyl resins, polyurethanes and polyesters, celluloses and cellulose-based derivatives, such as nitrocellulose, cellulose esters, such as cellulose acetate, cellulose acetopropionate or cellulose acetobutyrate, resins resulting from the condensation of formaldehyde with an arylsulfonamide, and mixtures thereof.

When the composition, such as a nail varnish, comprises an aqueous medium, then the film-forming polymer is generally present in the form of particles as a dispersion in the aqueous medium thus forming a latex or pseudolatex.

Among the film-forming polymers which can be used in aqueous medium, mention may be made of polyurethanes, for example anionic polyurethanes, polyester polyurethanes, polyether polyurethanes, free-radical polymers, in particular of acrylic, acrylic styrene and/or vinyl type, polyesters and alkyd resins, alone or as a mixture.

The dispersion can also comprise an associative polymer of polyurethane type or a natural gum, such as xanthan gum.

As polymers in aqueous dispersion, mention may be made of the dispersions of acrylic polymers sold under the names Neocryl XK-90®, Neocryl A-1070®, Neocryl A-1090®, Neocryl BT-62®, Neocryl A-1079® and Neocryl A-523® by the company Zeneca, and Dow Latex 432® by the company Dow Chemical. Use may also be made of aqueous dispersions of polyurethane, and in particular the polyester polyurethanes sold under the names “Avalure UR-405®”, “Avalure UR-410®”, “Avalure UR-425®” and “Sancure 2060®” by the company Goodrich and the polyether polyurethanes sold under the names “Sancure 878®” by the company Goodrich and “Neorez R-970®” by the company Avecia.

The film-forming polymer may be present in the composition according to the invention in an amount of solids which is effective for obtaining a film, generally ranging from 5% to 60% by weight relative to the total weight of the composition, and better still from 10% to 40% by weight.

As “organic solvents” which can be used in the composition of the invention, mention may be made, besides the hydrophilic organic solvents mentioned above, of ketones which are liquid at ambient temperature, such as methyl ethyl ketone, methyl isobutyl ketone, diisobutyl ketone, isophorone, cyclohexanone, acetone; ethers of propylene glycol which are liquid at ambient temperature, such as propylene glycol monomethyl ether, propylene glycol monomethyl ether acetate, dipropylene glycol, mono-n-butyl ether; short-chain esters (containing from 3 to 8 carbon atoms in total), such as ethyl acetate, methyl acetate, propyl acetate, n-butyl acetate, isopentyl acetate; ethers which are liquid at ambient temperature, such as diethyl ether, dimethyl ether or dichlorodiethyl ether; alkanes which are liquid at ambient temperature, such as decane, heptane, dodecane, isododecane, cyclohexane; aromatic cyclic compounds which are liquid at ambient temperature, such as toluene and xylene; aldehydes which are liquid at ambient temperature, such as benzaldehyde or acetaldehyde, and mixtures thereof.

In particular, in the case of a composition for the nails, such as a nail varnish, the organic solvent may be chosen from:
ketones which are liquid at ambient temperature, such as methyl ethyl ketone, methyl isobutyl ketone, diisobutyl ketone, isophorone, cyclohexanone or acetone;
alcohols which are liquid at ambient temperature, such as ethanol, isopropanol, diacetone alcohol, 2-butoxyethanol or cyclohexanol;
ethers of propylene glycol which are liquid at ambient temperature, such as propylene glycol monomethyl ether, propylene glycol monomethyl ether acetate or dipropylene glycol mono-n-butyl ether;
cyclic ethers such as γ-butyrolactone;
short-chain esters (containing from 3 to 8 carbon atoms in total), such as ethyl acetate, methyl acetate, propyl acetate, isopropyl acetate, n-butyl acetate, isopentyl acetate, methoxypropyl acetate or butyl lactate;
ethers which are liquid at ambient temperature, such as diethyl ether, dimethyl ether or dichlorodiethyl ether;
alkanes which are liquid at ambient temperature, such as decane, heptane, dodecane or cyclohexane;
alkyl sulfoxides such as dimethyl sulfoxide;
aldehydes which are liquid at ambient temperature, such as benzaldehyde or acetaldehyde;
ethyl 3-ethoxypropionate;
carbonates, such as propylene carbonate or dimethyl carbonate;
acetals such as methylal;
and mixtures thereof.

These solvents may be generally present in an amount ranging from 0 to 90%, preferably from 0.1 to 90%, more preferably from 10 to 90%, by weight relative to the total weight of the composition, and better still from 30 to 90%.

As “oils” which can be used in the compositions of the invention, mention may be made of: hydrocarbon-based oils of animal origin such as perhydrosqualene; hydrocarbon-based plant oils such as liquid triglycerides of fatty acids containing from 4 to 10 carbon atoms, for instance triglycerides of heptanoic acid or octanoic acid, or else sunflower oil, corn oil, soybean oil, grapeseed oil, sesame oil, apricot oil, macadamia oil, castor oil or avocado oil, triglycerides of caprylic/capric acids, jojoba oil, karite butter oil; linear or branched hydrocarbons of mineral or synthetic origin, such as paraffin oils and their derivatives, petroleum jelly, polydecenes, hydrogenated polyisobutene such as parleam; synthetic esters and ethers, in particular of fatty acids, such as, for example, purcellin oil, isopropyl myristate, 2-ethylhexyl palmitate, 2-octyldodecyl stearate, 2-octyldodecyl erucate, isostearyl isostearate; hydroxylated esters such as isostearyl lactate, octyl hydroxystearate, octyldodecyl hydroxystearate, diisostearyl malate, triisocetyl citrate, heptanoates, octanoates and decanoates of fatty alcohols; polyol esters such as propylene glycol dioctanoate, neopentyl glycol diheptanoate, diethylene glycol diisononanoate; pentaerythritol esters; fatty alcohols containing from 12 to 26 carbon atoms, such as octyldodecanol, 2-butyloctanol, 2-hexyldecanol, 2-undecylpentadecanol, oleyl alcohol; partially hydrocarbon-based and/or silicone-based fluoro oils; silicone oils such as volatile or nonvolatile, linear or cyclic polymethylsiloxanes (PDMSs) which are liquid or pasty at ambient temperature, such as cyclomethicones or dimethicones, optionally comprising a phenyl group, for instance phenyl trimethicones, phenyltrimethylsiloxydiphenylsiloxanes, diphenylmethyldimethyltrisiloxanes, diphenyl dimethicones, phenyl dimethicones or polymethylphenylsiloxanes; and mixtures thereof.

These oils can be present in an amount ranging from 0.01 to 90%, and better still from 0.1 to 85%, by weight relative to the total weight of the composition.

For the purpose of the present invention, the term “wax” is intended to mean a lipophilic compound solid at ambient temperature (25° C.) which has a reversible solid/liquid state transition and which has a melting point greater than or equal to 30° C. which can range up to 120° C.

The waxes can be hydrocarbon-based waxes, fluoro waxes and/or silicone waxes and can be of plant, mineral, animal and/or synthetic origin. In particular, the waxes have a melting point of greater than 25° C., and better still greater than 45° C.

As waxes which can be used in the composition of the invention, mention may be made of beeswax, carnauba wax or candelilla wax, paraffin, microcrystalline waxes, ceresin or ozokerite; synthetic waxes such as polyethylene waxes of Fischer-Tropsch waxes, waxes of silicones such as alkyl or alkoxy dimethicones containing from 16 to 45 carbon atoms.

As “plasticizers” which can be used in the compositions for nails according to the invention, mention may be made of glycols and their derivatives, glycol esters, propylene glycol derivatives, carboxylic acid esters, oxyethylenated derivatives, and mixtures thereof.

The content of plasticizer can, for example, range from 0.1% to 15% by weight relative to the total weight of the composition, and preferably from 0.5 to 10% by weight.

As “reducing agents” which can be used in the compositions for shaping or permanent reshaping of the hair, mention may, for example, be made of thioglycolic acid, glyceryl or glycyl monothioglycolate, cysteamine and its C₁-C₄ acylated derivatives, such as N-acetylcysteamine or N-propionylcysteamine, cysteine, N-acetylcysteine, N-mercaptoalkylamides of sugars, such as N-(mercapto-2-ethyl)gluconamide, beta-mercaptopropionic acid and its derivatives, thiolactic acid and its esters, such as glyceryl monothiolactate, thiomalic acid, pantetheine, thioglycerol, sulfites or biosulfites of an alkali metal or an alkaline-earth metal, the N-(mercaptoalkyl)-o-hydroxyalkylamides described in EP-354,835 and the N-mono- or N,N-dialkyl-4-mercaptobutyramides described in EP-368,763, the aminomercaptoalkylamides described in EP-403,267 and the alkylaminomercaptoalkylamides described in EP-432,000.

When the compositions according to the invention contain at least one reducing agent, said reducing agent is advantageously present at a maximum concentration of 20% by weight, and preferably of between 0.1 and 10% by weight, relative to the total weight of the composition.

As “fixing polymers” which can be used in the compositions for shaping and/or styling and/or maintaining the hair, mention may be made of anionic fixing polymers, cationic fixing polymers, nonionic fixing polymers, amphoteric fixing polymers, and mixtures thereof.

As examples of anionic fixing polymers, mention may be made of those containing carboxylic groups, such as the homo- or copolymers of acrylic or methacrylic acid or their salts, the copolymers of acrylic or methacrylic acid with a monoethylenic monomer such as ethylene, styrene, vinyl esters, acrylic or methacrylic acid esters optionally grafted onto a polyalkylene glycol such as polyethylene glycol, and optionally crosslinked, crotonic acid copolymers, copolymers of C₄-C₈ monounsaturated carboxylic acids or anhydrides, polyacrylamides comprising carboxylate groups.

As examples of cationic fixing polymers, mention may be made of:

(1) copolymers of acrylamide and of dimethylaminoethyl methacrylate in which the dimethylaminoethyl methacrylate is quaternized with dimethyl sulfate or with a dimethyl halide, copolymers of acrylamide and of methacryloyloxyethyltrimethylammonium chloride; the copolymer of acrylamide and of methacryloyloxyethyltrimethylammonium methosulfate; vinylpyrrolidone/dialkylaminoalkyl acrylate or methacrylate copolymers which may or may not be quaternized;

(2) cationic polysaccharides, preferably containing a quaternary ammonium, such as guar gums containing trialkylammonium cationic groups;

(3) quaternary copolymers of vinylpyrrolidone and of vinylimidazole;

(4) chitosans or their salts; the salts which can be used are in particular the acetate, lactate, glutamate, gluconate or pyrrolidonecarboxylate of chitosan;

(5) cationic cellulose derivatives such as copolymers of cellulose or of cellulose derivatives grafted with a water-soluble monomer comprising a quaternary ammonium.

The amphoteric fixing polymers which can be used according to the invention are chosen from the following polymers:

(1) copolymers containing acidic vinyl units and containing basic vinyl units;

(2) polymers comprising units derived:

a) from at least one monomer chosen from acrylamides or methacrylamides substituted on the nitrogen atom with an alkyl group,

b) from at least one acid comonomer containing one or more reactive carboxylic groups, and

c) from at least one basic comonomer such as esters, containing primary, secondary, tertiary and quaternary amine substituents, of acrylic and methacrylic acids, and the product of quaternization of dimethylaminoethyl methacrylate with dimethyl or diethyl sulfate.

Among the “conditioners or treating agents” which can be used in the compositions for styling or shaping the hair, use may in particular be made of volatile or nonvolatile, linear or cyclic silicones and mixtures thereof, polydimethylsiloxanes, quaternized polyorganosiloxanes, polyorganosiloxanes containing aminoalkyl groups modified with alkoxycarbonylalkyl groups, polyorganosiloxanes such as polydimethylsiloxane-polyoxyalkyl copolymer of the dimethicone copolyol type, a polydimethylsiloxane containing stearoxy end groups (stearoxy dimethicone), a polydimethylsiloxane-dialkylammonium acetate copolymer or a polydimethylsiloxane-polyalkylbetaine copolymer, polyorganosiloxanes modified with mercapto or mercaptoalkyl groups, such as those described in French Patent No. 1-530,369 and in European patent application No. 295,780, and also silanes such as stearoxytrimethylsilane.

As “surfactants” which can be used in the compositions of the invention, mention may be made of a surfactant of nonionic, anionic, cationic or amphoteric type. In particular, for the hair compositions, mention may be made of alkyl sulfates, alkylbenzene sulfates, alkyl ether sulfates, alkylsulfonates, quaternary ammonium salts, alkylbetaines, oxyethylenated alkylphenols, fatty acid alkanolamides, oxyethylenated fatty acid esters, and also other nonionic surfactants of the hydroxypropyl ether type.

The compositions according to the invention can also comprise one or more coloring materials chosen from water-soluble colorants, and pulverulent coloring materials such as pigments, pearlescent agents and glitter well known to those skilled in the art. The coloring materials can be present in the composition in an amount ranging from 0.01% to 50% by weight relative to the total weight of the composition, preferably from 0.01% to 30% by weight.

The term “pigments” should be understood to mean white or colored, mineral or organic particles of any shape, which are insoluble in the physiological medium and which are intended to color the composition.

The term “pearlescent agents” should be understood to mean iridescent particles of any shape, in particular produced by certain molluscs in their shell, or else synthesized.

The pigments can be white or colored and mineral and/or organic. Among mineral pigments, mention may be made of titanium dioxide, which has optionally been surface-treated, zirconium oxide or cerium oxide, and also zinc oxide, (black, yellow or red) iron oxide or chromium oxide, manganese violet, ultramarine blue, chromium hydrate and ferric blue, or metal powders such as aluminum powder or copper powder. Among organic pigments, mention may be made of carbon black, pigments of D & C type, lakes based on cochineal carmine or on barium, strontium, calcium or aluminum.

The pearlescent pigments may be chosen from white pearlescent pigments, such as mica coated with titanium or with bismuth oxychloride, colored pearlescent pigments such as titanium mica covered with iron oxides, titanium mica covered in particular with ferric blue or chromium oxide, titanium mica coated with an organic pigment of the abovementioned type, and also pearlescent pigments based on bismuth oxychloride.

The water-soluble colorants are, for example, beetroot juice and methylene blue.

The glitter can be chosen from those made of acrylic resin, polyester, polyethylene terephthalate, or of aluminum.

As “thickeners” which can be used according to the invention, mention may be made of cellulose and its derivatives such as cellulose ethers, heterobiopolysaccharides such as xanthan gum, scleroglucans, crosslinked or noncrosslinked polyacrylic acids.

Mention may be made, for example, of:
natural polymers such as xanthan and guar gums or else cellulose-based derivatives, starches and alginates;
synthetic crosslinked acrylic polymers such as the Carbopols® sold by the company Goodrich and the crosslinked and at least partially neutralized 2-acrylamido-2-methylpropanesulfonic acid (AMPS) polymers, such as, for example, the product sold under the name Hostacerin® AMPS by the company Clariant.

The thickeners are preferably present in proportions ranging between 0.1 and 5% by weight approximately, relative to the total weight of the composition.

The compositions according to the invention may also comprise one or more fillers, in particular in an amount ranging from 0.01% to 50% by weight relative to the total weight of the composition, preferably ranging from 0.01% to 30% by weight. The term “fillers” should be understood to mean colorless or white, mineral or synthetic particles of any shape, which are insoluble in the medium of the composition irrespective of the temperature at which the composition is manufactured. These fillers serve in particular to modify the rheology or the texture of the composition.

The fillers may be mineral or organic and of any shape, in platelet form, spherical or oblong, irrespective of the crystallographic shape (for example leaflet, cubic, hexagonal, orthorhombic, etc.). Mention may be made of talc, mica, silica, kaolin, powders of polyamide (Nylon®) (Orgasol® from Atochem), of poly-β-alanine and of polyethylene, powders of tetrafluoroethylene (Teflon®) polymers, lauroyllysine, starch, boron nitride, hollow polymer microspheres such as those made of polyvinylidene chloride/acrylonitrile, for instance Expancel® (Nobel Industrie), or of acrylic acid copolymers (Polytrap® from the company Dow Corning), and microbeads of silicone resin (Tospearls® from Toshiba, for example), particles of elastomeric polyorganosiloxanes, precipitated calcium carbonate, magnesium carbonate and magnesium hydrocarbonate, hydroxyapatite, hollow silica microspheres (Silica Beads® from Maprecos), glass or ceramic microcapsules, metal soaps derived from organic carboxylic acids containing from 8 to 22 carbon atoms, preferably from 12 to 18 carbon atoms, for example zinc stearate, magnesium stearate, lithium stearate, zinc laurate or magnesium myristate.

Of course, those skilled in the art will take care to choose this or these optional complementary compound(s), and/or its/their amount, in such a way that the advantageous properties of the corresponding composition according to the invention are not, or are not substantially, altered by the envisaged addition.

The compositions according to the invention may be provided in particular in the form of a suspension, a dispersion, a solution, a gel, or an emulsion, in particular an oil-in-water (O/W) or water-in-oil (W/O) or multiple (W/O/W or polyol/O/W or O/W/O) emulsion, or in the form of a cream, a paste, a mousse, a dispersion of vesicles, in particular of ionic or nonionic lipids, a two-phase or multiphase lotion, a spray or a paste.

For application to the hair in particular, the composition is generally in the form of aqueous, alcoholic or aqueous-alcoholic solutions; in the form of creams, lotions, gels, emulsions or mousses; or in the form of compositions for an aerosol, also comprising a pressurized propellant.

Those skilled in the art may choose the appropriate galenical form, and also the method for preparing it, on the basis of their general knowledge, taking into account, firstly, the nature of the constituents used, in particular their solubility in the carrier, and, secondly, the envisaged application for the composition.

The invention therefore relates in particular to a cosmetic composition for the care of and/or for making up the eyelashes, characterized in that it contains at least an effective amount of a bacterial extract as defined above, in a cosmetically acceptable medium comprising at least one film-forming polymer.

The composition may also comprise at least one cosmetic adjuvant chosen from oils, waxes, organic solvents, plasticizers, surfactants, fillers, pigments, agents which confer good curling properties on the eyelashes, and mixtures thereof. To promote the curling of the eyelashes, use may be made of a dispersion of multiphase particles as described in WO 03/007898, the multiphase particles comprising at least one soft phase which is at least partially external, comprising at least one soft polymer having at least a glass transition temperature of less than or equal to 60° C., and at least one rigid phase which is at least partially internal, the rigid phase being an amorphous material having at least a glass transition temperature of greater than 60° C., the soft polymer being attached at least partially by chemical grafting to the rigid phase.

According to a particular embodiment, the composition for the care of and/or for making up the eyelashes is characterized in that it is free of UV-screening agent. The expression “free of UV-screening agent” is intended to mean a composition which comprises less than 0.1% of UV-screening agent, preferably less than 0.01% of UV-screening agent, and even more preferably no UV-screening agent.

The composition will preferably be a mascara. The term “mascara” is intended to mean a composition intended to be applied to the eyelashes: it may be a make-up composition for the eyelashes, a make-up base for the eyelashes, a composition to be applied onto a mascara, also referred to as top-coat, or else a composition for cosmetic treatment of the eyelashes. The mascara is more particularly intended for human eyelashes, but also for false eyelashes.

The invention also relates to a cosmetic composition for the care of and/or for making up the nails, characterized in that it contains at least an effective amount of a bacterial extract as defined above in a cosmetically acceptable medium comprising water or a mixture of water and of hydrophilic organic solvents, and at least one film-forming polymer.

The composition may also comprise an agent chosen from a plasticizer, a surfactant, fillers, pigments, pearlescent agents, and mixtures thereof.

In particular, the composition may be: a make-up product for the nails, such as a colored nail varnish, a base for the nails, or “base-coat”, or a finishing composition, also called “top-coat”, to be applied under or over the make-up product for the nails, a nail varnish dissolver or else a cosmetic nailcare product such as a treating base intended to protect, to strengthen and/or to repair the nails.

The invention also covers a cosmetic composition for shaping and/or for styling the hair, characterized in that it contains at least an effective amount of a bacterial extract as defined above in a cosmetically acceptable medium comprising at least one agent chosen from a reducing agent and a fixing polymer.

In particular, the compositions for reshaping (shaping) the hair will comprise at least one reducing agent, and the compositions for styling the hair will comprise at least one fixing polymer.

The composition may also comprise at least one agent chosen from a surfactant, a film-forming polymer, a conditioner or treating agent, ceramides, a thickener, a propellant for packaging as lacquers, and mixtures thereof.

According to a particular embodiment, the composition for shaping and/or for styling the hair is characterized in that it is free of UV-screening agents. The expression “free of UV-screening agents” is intended to mean a composition which comprises less than 0.1% of UV-screening agent, preferably less than 0.01% of UV-screening agent, and even more preferably no UV-screening agent.

The composition for shaping and/or for styling the hair will generally be provided in the form of a hair composition for semi-permanent or permanent reshaping, or a shampoo or conditioner for permed hair, of a hairsetting gel or lotion, of a lotion for blow-drying, or of a setting and styling composition such as mousses, lacquers or sprays, or else in the form of a mascara or of a make-up lacquer for hair.

Preferably, the compositions of the invention comprise a cell extract of a nonfruiting nonphotosynthetic filamentous bacterium, as defined above.

Preferably, the compositions according to the invention are characterized in that the bacterial extract or a fraction of said extract is in an amount of between 0.01% and 100% of the total weight of the composition, preferably between 0.05 and 10% of the total weight of the composition, and even more preferably between 0.1 and 5% of the total weight of the composition.

The application of said compositions will be carried out by any suitable means, such as a nail varnish brush, a brush, a spray or the fingers, for example.

When the composition is in the form of a nailcare solution, the application may be carried out by simply soaking the fingers in said solution.

Another feature of the present invention is a cosmetic process for shaping and/or styling the hair, or for the care of and/or for making-up the eyelashes or the nails, characterized in that one of the compositions as defined above is applied to the hair and/or the eyelashes and/or the nails.

The compositions according to the invention will be applied according to the usual technique for using said compositions. For example:
the application to hair can take place before (lotion, 1 hour before), during (shampoo) or after (lotions, sprays) shampooing; the composition may be applied to dry hair (lacquers, sprays, lotions) or to wet hair (permanent-reshaping or setting compositions);
the application of a care base to the nails may take place before or after the varnish has been applied;
the cosmetic composition for treating the eyelashes may be applied just after application of the curling mascara; advantageously, the bacterial extract will be a component of the curling mascara itself.

The compositions may be applied daily or at a frequency of 2 to 3 applications per week, for a period of 1 to 3 months, renewable according to the degree of alteration of the keratin materials of the individual to be treated. The invention is illustrated in greater detail in the nonlimiting examples which follow.

The accompanying figure of drawing represents the length of suspended locks after treatment with the bacterial extract according to the invention, as a function of time (hours).

In order to further illustrate the present invention and the advantages thereof, the following specific examples are given, it being understood that same are intended only as illustrative and in nowise limitative. In said examples to follow, all parts and percentages are given by weight, unless otherwise indicated.

### EXAMPLES

### Example 1

### Preparation of the Extracts of Vitreoscilla filiformis

The Vitreoscilla filiformis strain (ATCC 15551) is cultured according to the method described in WO-A-93-00741. This is a continuous culturing method. The culturing is carried out at 26° C. for at least 48 hours, until a suitable cell concentration corresponding to an optical density at 600 nm of greater than or equal to 1.5 is obtained. The strain is subcultured at 2% V/V in fresh medium for approximately 48 hours until a stable culture is obtained. A 1 litre Erlenmeyer flask containing 200 ml of fresh medium is then seeded with 4 ml of the preceding culture. Culturing in the Erlenmeyer flask is carried out at 26° C. on a culture platform shaken at 100 rpm. The stock sediment thus obtained serves as inoculum for a 50 litre fermenter. Growth takes place at 26° C., pH 7, 100 rpm and pO₂>15%.

After growth for 30 hours, the biomass is transferred into a fermenter with a working capacity of 3000 litres, in order to be cultured under the same conditions. After growth for 48 hours, the cells are continuously harvested. The biomass is then concentrated approximately 50-fold by centrifugation. The cells obtained are then frozen as the continuous culture progresses. These cells can be used as they are, after thawing (nonstabilized cell extract), or else can be stabilized by autoclaving at 121° C. for 20 to 40 minutes (stabilized cell extract). The cells are then ruptured during the sterilization, releasing the cytosol and aggregating the proteins and the walls. The product obtained is then a two-phase product.

The liquid supernatant phase can be filtered through 0.22 μm so as to remove the particles (“supernatant”).

The bacterial extract, in the form of cell extract (stabilized or nonstabilized) or of supernatant, can be used as it is (aqueous form) or can be lyophilized according to conventional techniques (lyophilized form).

### Example 2

### Demonstration of a Nail-strengthening and -embellishing Effect: Improvement of the Hardness, of the Growth Rate, of the Thickness and of the General Appearance of the Nails

An extract of Vitreoscilla filiformis as prepared according to Example 1 (cell extract in aqueous form) is applied in a pure or diluted (for example: ½, 1/10 and 1/20) form to the nails for 1 to 5 minutes at ambient temperature. The application is carried out by soaking the nails in the extract or by application using a nail varnish brush or else by application using a roll-on applicator, or the like. One hand is treated and the other hand is not treated: the latter constitutes the control. It is important not to rinse off the product before one hour after application.

As early as the first applications, a nail-strengthening effect is noted in the presence of the bacterial extract: the nails are described as harder and more resistant to breaking and/or to splitting compared to the control hand.

After several applications, an improvement in the condition of the nails is observed.

These results were confirmed by a study carried out on 30 individuals: 15 of them have fragile and soft nails, the other 15 have hard and brittle nails.

The treatment was carried out on only one hand, the other constituting the control.

The nails of the treated hand were soaked for 1 minute in the aqueous solution of cell extract of Vitreoscilla filiformis in pure form as prepared in Example 1, without rinsing for at least 30 minutes to 1 hour after application.

The application was repeated daily for a period of 3 weeks, at the end of which a first evaluation (D21) was performed, and the treatment was then carried out again for 3 weeks with a second evaluation at the end of the 6 weeks (D42).

These evaluations consisted in:
measuring the growth rate of the treated nail compared to the control nail;
measuring the thickness of the treated nail compared to the control nail;
scoring from 0 (− effect) to 9 (++ effect) the effectiveness of the product according to various criteria: hardness, brilliance, smooth appearance, split, nails, brittle nails, general condition of the nail.

For the measurements of growth rate and of thickness of the nails, the statistical analysis is carried out using the Student's t test.

For the subjective evaluation of the general appearance of the nail, the test used is the Wilcoxon signed-rank test.

a) Growth rate:

The growth rate of the nails is measured before and after 3 and 6 weeks (D21 and D42) of daily use of the product, by analysis of images taken using a video microscope.

The video microscope is a variable-objective fiber-optic mobile microscope coupled to a computer system for image acquisition (Puppin D et al., Journal of the American Academy of Dermatology, 1993, 28: 923-927). The objective is placed directly in front of the area studied, without contact, and the image is observed on the computer screen.

To evaluate the growth rate of the nail, the layer is marked with a small striation at its surface on D0. The progression of this striation on the nail makes it possible to calculate a growth rate.

The tables below show a summary of the individual results obtained:

Nail growth rate (mm/day)

| | Treated | Nontreated |
|---|---|---|
| D21 | 0.133 ± 0.005 | 0.134 ± 0.004 |
| D42 | 0.132 ± 0.004 | 0.129 ± 0.004 |

Variations

| | D21 | D42 |
|---|---|---|
| % variation | 0% | 4% |
| % of individuals showing an | 43% | 66% |
| improvement | | |

The growth rate of the nails is therefore increased after 21 days on 43% of the individuals, and after 42 days on 66% of the individuals, compared to a nontreated nail.

b) Nail thickness:

The thickness of the nail is measured by ultrasound echography using DermaScan C® version 3 before and after 6 weeks (D42) of daily use of the product. The principle is as follows: an ultrasound beam is emitted by a piezoelectric ceramic and this beam is partially reflected by the interfaces separating two media of different ultrasound impedance (Wollina U. et al., Skin research and Technology, 2001, 7: 60-64).

This method allows a two-dimensional evaluation of the nail and image analysis makes it possible to calculate the thickness of the nail with a precision estimated at 2%.

The tables below show a summary of the individual results obtained:

Thickness of the nails (mm)

| | Treated | Nontreated |
|---|---|---|
| D0 | 0.441 ± 0.009 | 0.440 ± 0.009 |
| D42 | 0.465 ± 0.010 | 0.441 ± 0.009 |

Variations

| | D42 |
|---|---|
| % variation | 5% |
| % of individuals showing an | 79% |
| improvement | |

The product significantly increases the thickness of the nails on 79% of the individuals at D42, compared to the nontreated nail.

c) General appearance of the nail:

The effectiveness of the product at D0, D5, D21 and D42 is evaluated by the individuals on the basis of assessment (structured scale from 0 to 9) of the following criteria: hardness, brilliance, smooth appearance, split nails, brittle nails, and general condition of the nail.

The table below shows a summary of the variations of the results given by the volunteers:

| | D5 | D21 | D42 |
|---|---|---|---|
| Hardness of the nail | 0.5 ± 0.3 | 0.8 ± 0.3 | 1.4 ± 0.5 |
| Brilliance of the nails | 0.7 ± 0.2 | 1.3 ± 0.3 | 1.8 ± 0.4 |
| Smooth appearance of the nails | 0.8 ± 0.2 | 0.8 ± 0.3 | 1.3 ± 0.4 |
| Split nails | 1.4 ± 0.5 | 1.8 ± 0.7 | 2.9 ± 0.6 |
| Brittle nails | 1.8 ± 0.5 | 2.2 ± 0.7 | 3.6 ± 0.5 |
| General condition of the nails | 1.1 ± 0.3 | 2.2 ± 0.5 | 2.6 ± 0.5 |

After 42 days of treatment, the individuals noted a clear improvement in the condition of their nails: they were harder, more brilliant, smoother, less split, less brittle and less damaged. The improvement was noted as early as the first 5 days and continued until the end of treatment.

All these data show that the cell extract of Vitreoscilla filiformis makes it possible to strengthen the nails, increasing their hardness, their growth rate, their thickness and their overall appearance (smoother, more brilliant, less split, less brittle and less damaged).

### Example 3

### Demonstration of an Effect on the Elasticity and the Shape of the Hair

Standardized locks of natural hair (length 30 cm, mass 2.5 g) were immersed for 10 minutes at ambient temperature (approx. 21° C.) in 5 ml of water (control) or of extract of Vitreoscilla filiformis (pure cell extract or cell extract diluted: ½, 1/10 and 1/20).

The locks were then rinsed in a standardized manner: the fingers were passed along the locks 15 times under a continuous stream of tap water at 30° C.

The locks were then rolled on conventional rollers (diameter=2 cm; length 10 cm), and then left to dry under a hairdryer (t=65° C.) for 30 minutes.

Once the locks had been unrolled from the rollers, they were then suspended vertically and the total length of each one was recorded at t0 and then at various time intervals, for example 3 and 19 hours, in order to quantify the rate of relaxation of these locks (return to the prior state).

A treated lock which thus keeps the shortest length for the longest possible time thus gives an account of the effect of hold and of shaping of the hair provided by the product studied.

The results of lock lengths (cm) measured after treatment are given in the table below and represented in graph form in the attached figure of drawing.

HOURS

| | 0 | 3 | 19 |
|---|---|---|---|
| Control | 11.5 | 15.5 | 17 |
| Pure 71569 | 11 | 14.5 | 16 |
| 71569 diluted 1/2 | 11.5 | 15 | 16 |
| 71569 diluted 1/10 | 11.5 | 15 | 16 |
| 71569 diluted 1/20 | 11.5 | 14.5 | 15.5 |

These results show that the cell extract of Vitreoscilla filiformis applied pure or diluted to a lock of hair confers on it a better hold compared to a control (water): it makes it possible to prolong the curled effect of a lock of hair, by increasing its elasticity and maintaining its shaping. This effect is not dose-dependent, the result being substantially equivalent in the presence of the pure extract or of an extract diluted 1/20.

The same experiment was reproduced with the supernatant from said cell extract: the results also show an effect on the hold of the curled effect of the lock compared to the control (water).

### Example 4

### Examples of Formulations

The values are expressed as % relative to the total weight of the composition, unless otherwise indicated.

These formulations are prepared according to the usual methods.

Nail-strengthening care base:

| | |
|---|---|
| Nitrocellulose | 15% |
| Plasticizer and resin | 9% |
| Cell extract of Vitreoscilla filiformis | 1% |
| (according to Example 1) | |
| Ethyl alcohol | 7% |
| Isopropyl alcohol | 7% |
| Water | 2% |
| Butyl acetate | 30% |
| Ethyl acetate | qs 100% |

Non-rinsed-out lotion for permanent-waved hair:

| | |
|---|---|
| Cell extract of Vitreoscilla filiformis | 1% |
| (according to Example 1) | |
| Linoleic acid | 1% |
| Propylene glycol | 22.8% |
| 95° ethanol | 55.1% |
| Purified water | qs 100% |

Mascara:

| | | |
|---|---|---|
| Supernatant extracted from Vitreoscilla | 1 | g |
| filiformis (lyophilized form) | | |
| Carnauba wax | 20 | g |
| Polyoxyethylenated (30 EO) glyceryl | 8 | g |
| stearate (Tagat S from the company | | |
| Goldschmidt) | | |
| Black iron oxide | 5 | g |
| Propylene glycol | 5 | g |
| Hydroxyethylcellulose | 2.5 | g |
| Water | qs 100 | g |

Similar compositions may be prepared with 5% of extract.

Each patent, patent application, publication and literature article/report cited or indicated herein is hereby expressly incorporated by reference.

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

1. A cosmetic composition comprising an effective amount of at least one extract of at least one nonfruiting nonphotosynthetic filamentous bacterium for improving the structure of keratin materials, formulated into a topically applicable, cosmetically acceptable medium therefor that is compatible with said keratin materials.

2. The cosmetic composition as defined by claim 1, comprising an amount of said at least one extract of at least one nonfruiting nonphotosynthetic filamentous bacterium effective for hardening the nails and/or increasing their resistance to breaking and/or to splitting.

3. The cosmetic composition as defined by claim 1, comprising an amount of said at least one extract of at least one nonfruiting nonphotosynthetic filamentous bacterium effective for increasing the thickness of the nails.

4. The cosmetic composition as defined by claim 1, comprising an amount of said at least one extract of at least one nonfruiting nonphotosynthetic filamentous bacterium effective for stimulating the growth and/or regrowth of the nails.

5. The cosmetic composition as defined by claim 1, comprising an amount of said at least one extract of at least one nonfruiting nonphotosynthetic filamentous bacterium effective for prolonging the retention of a nail varnish.

6. The cosmetic composition as defined by claim 1, comprising an amount of said at least one extract of at least one nonfruiting nonphotosynthetic filamentous bacterium effective for increasing the elasticity and/or maintaining the shaping of the hair.

7. The cosmetic composition as defined by claim 1, comprising an amount of said at least one extract of at least one nonfruiting nonphotosynthetic filamentous bacterium effective for prolonging the curling of the hair.

8. The cosmetic composition as defined by claim 1, comprising an amount of said at least one extract of at least one nonfruiting nonphotosynthetic filamentous bacterium effective for increasing the elasticity and/or maintaining the shaping of the eyelashes.

9. The cosmetic composition as defined by claim 1, comprising an amount of said at least one extract of at least one nonfruiting nonphotosynthetic filamentous bacterium effective for prolonging the curling of the eyelashes.

10. The cosmetic composition as defined by claim 1, said at least one bacterium belonging to the genus Beggiatoa, Vitreoscilla, Flexithrix or Leucothrix.

11. The cosmetic composition as defined by claim 10, said at least one bacterium being a Vitreoscilla filiformis strain.

12. The cosmetic composition as defined by claim 11, said at least one bacterium being the ATCC15551 strain.

13. The cosmetic composition as defined by claim 1, said at least one extract comprising a cell extract or a supernatant from said extract.

14. The cosmetic composition as defined by claim 13, said at least one extract comprising a cell extract or active fraction thereof.

15. The cosmetic composition as defined by claim 1, further comprising at least one film-forming polymer.

16. The cosmetic composition as defined by claim 1, comprising water or a mixture of water and at least one hydrophilic organic solvent, and at least one film-forming polymer.

17. The cosmetic composition as defined by claim 1, further comprising a reducing agent.

18. The cosmetic composition as defined by claim 1, further comprising a fixing polymer.

19. The cosmetic composition as defined by claim 1, comprising a nail varnish.

20. The cosmetic composition as defined by claim 1, comprising a suspension, a dispersion, a solution, a gel, an emulsion, a cream, a paste, a mousse, a vesicular dispersion, a lotion, or a spray.

21. The cosmetic composition as defined by claim 1, comprising a mascara.

22. The cosmetic composition as defined by claim 1, comprising a hair composition.

23. The cosmetic composition as defined by claim 1, said at least one bacterial extract or active fraction thereof comprising from 0.01% to 100% by weight thereof.

24. The cosmetic composition as defined by claim 1, said at least one bacterial extract or active fraction thereof comprising from 0.05% to 10% by weight thereof.

25. The cosmetic composition as defined by claim 1, said at least one bacterial extract or active fraction thereof comprising from 0.1% to 5% by weight thereof.

26. A cosmetic treatment for improving the structure of a keratin material, comprising topically applying thereon an effective amount of the cosmetic composition as defined by claim 1.

27. A cosmetic treatment for hardening the nails and/or increasing their resistance to breaking and/or to splitting, comprising topically applying thereon an effective amount of the cosmetic composition as defined by claim 2.

28. A cosmetic treatment for increasing the thickness of the nails, comprising topically applying thereon an effective amount of the cosmetic composition as defined by claim 3.

29. A cosmetic treatment for stimulating the growth and/or regrowth of the nails, comprising topically applying thereon an effective amount of the cosmetic composition as defined by claim 4.

30. A cosmetic treatment for prolonging the retention of a nail varnish, comprising topically applying thereon an effective amount of the cosmetic composition as defined by claim 5.

31. A cosmetic treatment for increasing the elasticity and/or maintaining the shaping of the hair, comprising topically applying thereon an effective amount of the cosmetic composition as defined by claim 6.

32. A cosmetic treatment for prolonging the curling of the hair, comprising topically applying thereon an effective amount of the cosmetic composition as defined by claim 7.

33. A cosmetic treatment for increasing the elasticity and/or maintaining the shaping of the eyelashes, comprising topically applying thereon an effective amount of the cosmetic composition as defined by claim 8.

34. A cosmetic treatment for prolonging the curling of the eyelashes, comprising topically applying thereon an effective amount of the cosmetic composition as defined by claim 9.
